(19) United States
(12) Patent Application Publication
(10) Pub. No.: US 2021/0078932 A1
(43) Pub. Date: Mar. 18, 2021

(54) 3,4-DIHYDROXYPHENETHYL 3-HYDROXYBUTANOATE, PREPARATION AND USE THEREOF

(71) Applicants: XI'AN JIAOTONG UNIVERSITY, Xi'an (CN); CHENGDU MEDICAL COLLEGE, Chengdu (CN)

(72) Inventors: JIAN-GANG LONG, Xi'an (CN); YA-CHONG HU, Xi'an (CN); YONG-YAO WANG, Xi'an (CN); ZHEN WANG, Xi'an (CN); QING-QING MA, Xi'an (CN); YU-XIA ZHANG, Xi'an (CN); QING-LIN JIANG, Chengdu (CN); XIAO-HONG XU, Chengdu (CN); TING-HUA ZHANG, Chengdu (CN); JIAN-KANG LIU, Xi'an (CN)

(21) Appl. No.: 17/013,539

(22) Filed: Sep. 5, 2020

(30) Foreign Application Priority Data
Sep. 17, 2019 (CN) 201910875705.0

(51) Int. Cl. C07C 69/612; C07C 67/03; C07C 41/09; C07C 43/205

(52) U.S. Cl. CPC C07C 69/612; C07C 43/2055; C07C 41/09; C07C 67/03

(57) ABSTRACT

The present disclosure discloses a novel compound, 3,4-dihydroxyphenethyl 3-hydroxybutanoate, a method for preparing the same and use of the same, and in particular, a compound of formula I, use of the compound of formula I, optically pure isomers of the compound, a mixture of enantiomers in any ratio, or pharmaceutically acceptable salts thereof in preparing health food and drug for relieving brain fatigue, improving learning and memory abilities, and ameliorating mania mood related to brain fatigue.

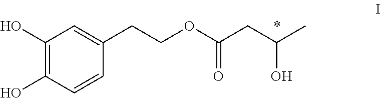 [SMILES: CC(O)CC(=O)OCCc1ccc(O)c(O)c1]

### CROSS-REFERENCE TO RELATED APPLICATION

This application claims all benefits accruing under 35 U.S.C. § 119 from China Patent Application No. 201910875705.0, filed on Sep. 17, 2019 in the China National Intellectual Property Administration, the content of which is hereby incorporated by reference.

### TECHNICAL FIELD

The present disclosure relates to the biological and pharmaceutical field, in particular to 3,4-dihydroxyphenethyl 3-hydroxybutanoate which is a novel compound, a method for preparing the same, and use of the same in preparing health foods and drugs for relieving brain fatigue.

### BACKGROUND

In modern society where lifestyles are becoming increasingly busy, more and more people suffer from brain fatigue induced by long-term and high-intensity work or high-stress environments. Brain fatigue in different levels can impair memory acquisition and consolidation, lead to decreased attention and alertness, and cause mental symptoms such as mania and depression. Studies have shown that sleep can regulate neuron function of the brain for storing memories. Sleep deprivation can have a significant negative effect on various functions of human and animal bodies, especially on aspects of cognition, memory, and emotion. The brain fatigue model established using the sleep deprivation method is a classic animal model for studying brain fatigue.

Studies have found that hydroxytyrosol (HT), with a molecular formula of C₈H₁₀O₃ and a relative molecular weight of 154.16, is a very effective mitochondrial nutrient with effects of anti-inflammation, anti-oxidation, and delaying neurodegenerative diseases. On one hand, HT, rich in hydroxyl groups, has certain reducibility, can react with excessive free radicals in cells, and can reduce the DNA damage caused by oxidative stress, thereby improving the function of mitochondria; on the other hand, HT can also activate mitochondrial biosynthesis to increase the number of healthy mitochondria, thereby reducing the proportion of damaged mitochondria, and protecting the physiological function of cells. Studies have shown that HT has an effect of protecting mitochondria from oxidative damage caused by exercise fatigue. However, there is no research report related to this compound on brain fatigue currently.

β-hydroxybutyric acid (β-HB), with a molecular formula of C₄H₈O₃ and a relative molecular weight of 104, is a kind of ketone bodies. In the case where sugar is insufficiently supplied, the liver generates a large amount of ketone bodies for supplying energy to peripheral tissues. As β-HB accounts for about 70% of the total amount of ketone bodies, it is generally considered to be the main energy supplying substance exported by the liver to peripheral tissues. In addition to energy supply, β-HB can also act as an endogenous biologically active small molecule, which plays an important role in protecting nerves, heart and blood vessels, and other tissues and organs. Therefore, β-HB can function as an important energy supplying substance for external supplementation. However, there is no research report related to this compound on brain fatigue currently.

### SUMMARY

An object of the present disclosure is to provide a novel compound, 3,4-dihydroxyphenethyl 3-hydroxybutanoate (also named hydroxytyrosol hydroxybutyrate, HT-HB), a method for preparing the same, a pharmaceutical composition containing the compound, and a method for relieving brain fatigue, specifically for improving learning and memory abilities, or ameliorating mania related to brain fatigue.

The present disclosure provides a compound having a chemical structure represented by formula I, wherein * indicates a chiral carbon.

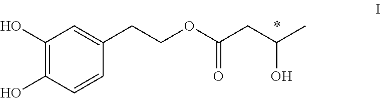 [SMILES: CC(O)CC(=O)OCCc1ccc(O)c(O)c1]

The present disclosure provides an embodiment of a method for preparing the compound represented by formula I, including:

S1, synthesizing β-benzyloxybutyric acid;

S2, synthesizing 3,4-dibenzyloxyphenylethanol;

S3, synthesizing 3,4-bis(benzyloxy)phenethyl 3-(benzyloxy)butanoate; and

S4, synthesizing 3,4-dihydroxyphenethyl 3-hydroxybutanoate.

In an embodiment, the step S1 includes reacting crotonic acid with benzyl alcohol to synthesize β-benzyloxybutyric acid.

In an embodiment, the step S2 includes reacting 3,4-dihydroxyphenylethanol with benzyl bromide to synthesize 3,4-dibenzyloxyphenylethanol.

In an embodiment, the step S3 includes reacting β-benzyloxybutyric acid synthesized in the step S1 with 3,4-dibenzyloxyphenylethanol synthesized in the step S2 to synthesize 3,4-bis(benzyloxy)phenethyl 3-(benzyloxy)butanoate.

In an embodiment, the step S4 includes reacting 3,4-bis(benzyloxy)phenethyl 3-(benzyloxy)butanoate synthesized in the step S3 with anhydrous methanol.

In an embodiment, the step S1 includes:

weighing and placing crotonic acid in a first reaction vessel, to which benzyl alcohol and mercuric acetate are sequentially added to form a first mixture, and stirring the first mixture at room temperature overnight;

cooling the first reaction vessel to about 0° C., adding sodium hydroxide within about 5 minutes to about 10 minutes to the first reaction vessel, then adding a sodium hydroxide water solution containing sodium borohydride to the first reaction vessel, and keeping the first reaction vessel at about 0° C. for about 3 minutes to about 10 minutes;

stirring the first mixture at room temperature for about 1 hour to about 2 hours and then filtering to obtain a filtrated liquid;

extracting the filtrated liquid to remove excess benzyl alcohol; and

acidifying the filtrated liquid to a pH value of about 2 to precipitate β-benzyloxybutyric acid.

In an embodiment, the step S2 includes:

weighing and placing 3,4-dihydroxyphenylethanol and potassium carbonate in a second reaction vessel, to which anhydrous acetone and benzyl bromide are sequentially added to form a second mixture; and

stirring the second mixture at about 70° C. under reflux to react for about 4 hours to about 5 hours.

In an embodiment, the step S3 includes:

weighing and placing β-benzyloxybutyric acid synthesized in the step S1 in a third reaction vessel, to which tetrahydrofuran, 3,4-dibenzyloxyphenylethanol synthesized in the step S2, 1-ethyl-3-(3-dimethylaminopropyl)carbodiimide, and 4-dimethylaminopyridine are added to form a third mixture; and

stirring the third mixture at about 30° C. for about 3 hours to 4 hours.

In an embodiment, the step S4 includes:

weighing and placing 3,4-bis(benzyloxy)phenethyl 3-(benzyloxy)butanoate synthesized in the step S3 in a fourth reaction vessel, to which anhydrous methanol and palladium on carbon catalyst are sequentially added to form a fourth mixture; and

stirring the fourth mixture at room temperature in hydrogen gas atmosphere for about 16 hours.

In an embodiment of step S1, crotonic acid is weighed and placed in a reaction vessel, to which benzyl alcohol and mercuric acetate are sequentially added to form a mixture. The mixture is stirred at room temperature overnight. The reaction vessel is placed in a low-temperature condensation tank and cooled to 0° C., added with sodium hydroxide (3N purity) within 5 to 10 minutes, and then added with a sodium hydroxide (3N purity) water solution containing 0.5 M of sodium borohydride, followed by keeping the mixture at 0° C. for 3 to 10 minutes. After that, the reaction vessel is taken out from the tank, and the mixture is stirred at room temperature for 1 to 2 hours and then filtered to obtain a filtrated liquid, which is extracted with ethyl ether 3 to 4 times to remove excess benzyl alcohol. The filtrated liquid is acidified by 10% (mass percentage) hydrochloric acid to a pH value of 2 to precipitate a large amount of white solid, which is filtered out. The white solid is β-benzyloxybutyric acid.

In an embodiment of step S2, 3,4-dihydroxyphenylethanol and potassium carbonate are weighed and placed in a reaction vessel, to which anhydrous acetone and benzyl bromide are sequentially added to form a mixture. The mixture is stirred at 70° C. under reflux to react for 4 to 5 hours. Once the completeness of the reaction is confirmed by thin layer chromatography (TLC), the reaction product is filtered to remove the potassium carbonate, and is concentrated and then applied to column chromatography to obtain 3,4-dibenzyloxyphenylethanol.

In an embodiment of step S3, β-benzyloxybutyric acid prepared in S1 is weighed and placed in a reaction vessel, to which tetrahydrofuran (THF) is added and dissolved. Then, 3,4-dibenzyloxyphenylethanol prepared in S2, 1-ethyl-3-(3-dimethylaminopropyl)carbodiimide (EDCI), and 4-dimethylaminopyridine (DMAP) are added into the reaction vessel to form a mixture. The mixture is stirred and reacted while the reaction vessel is in an oil bath at 30° C. for 3 to 4 hours. The reaction is terminated once disappearance of 3-benzyloxybutyric acid is confirmed by TLC. The reaction product is concentrated, and then dissolved and washed with ethyl acetate (EA) 2 to 3 times to obtain a solution, which is concentrated and applied to column chromatography to obtain 3,4-bis(benzyloxy)phenethyl 3-(benzyloxy)butanoate.

In an embodiment of S4, 3,4-bis(benzyloxy)phenethyl 3-(benzyloxy)butanoate prepared in S3 is weighed and placed in a reaction vessel, to which anhydrous methanol and 10 wt % palladium on carbon catalyst (Pd/C) are sequentially added, followed by stirring at room temperature in hydrogen gas atmosphere for 16 hours to have a reaction. The reaction product is filtered, concentrated, and applied to column chromatography to obtain 3,4-dihydroxyphenethyl 3-hydroxybutanoate.

In some embodiments, a ratio of crotonic acid, benzyl alcohol, mercuric acetate, sodium hydroxide, sodium hydroxide water solution in S1 is 1 mmol to 30 mmol:1 ml to 300 ml:1 mmol to 300 mmol:1 ml to 300 ml:1 ml to 300 ml, and in an embodiment is 29.7 mmol:30 ml:30 mmol:30 ml:30 ml.

In some embodiments, a ratio of 3,4-dihydroxyphenylethanol, potassium carbonate, anhydrous acetone, and benzyl bromide in S2 is 1 mmol to 20 mmol:1 mmol to 100 mmol:1 ml to 200 ml:1 mmol to 80 mmol, and in an embodiment is 6.49 mmol:25.9 mmol:20 ml:13.62 mmol.

In some embodiments, a ratio of β-benzyloxybutyric acid, THF, 3,4-dibenzyloxyphenylethanol, EDCI, DMAP in S3 is 1 mmol to 15 mmol:1 ml to 300 ml:1 mmol to 20 mmol:1 mmol to 60 mmol:1 mg to 500 mg, and in an embodiment is 6.4 mmol:45 ml:4 mmol:8 mmol:50 mg.

In some embodiments, a ratio of 3,4-bis(benzyloxy)phenethyl 3-(benzyloxy)butanoate, anhydrous methanol, and 10 wt % Pd/C in S4 is 1 mmol to 20 mmol:1 ml to 400 ml:1 mg to 1000 mg, and in an embodiment is 3.92 mmol:40 ml:200 mg, and the pressure of the hydrogen gas is one atmospheric pressure.

The present disclosure further provides another embodiment of the method for preparing the compound represented by formula I, including:

S1′, synthesizing 3,4-dibenzyloxyphenylethanol;

S2′, synthesizing 3-oxobutanoic acid;

S3′, synthesizing 3,4-bis(benzyloxy)phenethyl 3-oxobutanoate;

S4′, synthesizing 3,4-dihydroxyphenethyl 3-oxobutanoate; and

S5′, synthesizing 3,4-dihydroxyphenethyl 3-hydroxybutanoate.

In an embodiment, the step S1′ includes synthesizing 3,4-dibenzyloxyphenylethanol from 3,4-dihydroxyphenylethanol.

In an embodiment, the step S2′ includes synthesizing 3-oxobutanoic acid from ethyl acetoacetate.

In an embodiment, the step S3′ includes reacting 3,4-dibenzyloxyphenylethanol synthesized in the step S1′ and 3-oxobutanoic acid synthesized in the step S2′ to synthesize 3,4-bis(benzyloxy)phenethyl 3-oxobutanoate.

In an embodiment, the step S4′ includes synthesizing 3,4-dihydroxyphenethyl 3-oxobutanoate from 3,4-bis(benzyloxy)phenethyl 3-oxobutanoate synthesized in the step S3′.

In an embodiment, the step S5′ includes synthesizing 3,4-dihydroxyphenethyl 3-hydroxybutanoate from 3,4-dihydroxyphenethyl 3-oxobutanoate synthesized in the step in S4′.

In an embodiment, the step S1′ includes:

weighing and placing 3,4-dihydroxyphenylethanol and potassium carbonate in a first reaction vessel, to which anhydrous acetone and benzyl bromide are sequentially added to form a first mixture; and

stirring the first mixture at about 70° C. under reflux for about 4 hours to about 5 hours.

In an embodiment, the step S2′ includes:

weighing and placing ethyl acetoacetate in a second reaction vessel, to which NaOH water solution is added; and

placing the second reaction vessel in oil bath at about 60° C. for about 3 hours to have a reaction.

In an embodiment, the step S3′ includes:

weighing and placing 3,4-dibenzyloxyphenylethanol synthesized in the step S1′ and the 3-oxobutanoic acid synthesized in the step S2′ in a third reaction vessel, to which dichloromethane, 1-ethyl-3-(3-dimethylaminopropyl)carbodiimide, and 4-dimethylaminopyridine are sequentially added to a mixed solution;

reacting the mixed solution at room temperature for about 1 hour.

In an embodiment, the step S4′ includes:

weighing and placing 3,4-bis(benzyloxy)phenethyl 3-oxobutanoate synthesized in the step S3′ in a fourth reaction vessel, to which methanol is added, followed by ultrasonic dissolving to form a solution;

adding palladium on carbon catalyst to the solution under a protection of argon gas, followed by replacing air in the fourth reaction vessel with hydrogen gas; and

reacting the solution at room temperature for about 2 hours.

In an embodiment, the step S5′ includes:

weighing and placing 3,4-dihydroxyphenethyl 3-oxobutanoate synthesized in the step S4′ in a fourth reaction vessel, to which absolute ethanol is added, followed by ultrasonic dissolving; and

placing the fourth reaction vessel at about 0° C. and adding NaBH₄.

In an embodiment of step S1′, 3,4-dihydroxyphenylethanol and potassium carbonate are weighed and placed in a reaction vessel, to which anhydrous acetone and benzyl bromide are sequentially added to form a mixture. The mixture is stirred and reacted at 70° C. under reflux 4 to 5 hours. Once the completeness of the reaction is confirmed by TLC, the reaction product is filtered to remove the potassium carbonate, and is concentrated and then applied to column chromatography to obtain 3,4-dibenzyloxyphenylethanol.

In an embodiment of step S2′, ethyl acetoacetate is weighed and placed in a reaction vessel, to which freshly prepared NaOH (1N purity) water solution is added, followed by placing the reaction vessel in oil bath at 60° C. for 3 hours to have a reaction. Once the completeness of the reaction is confirmed by TLC, the reaction solution is cooled to room temperature, and then the reaction vessel is placed in an ice bath environment at 0° C. 10% (mass percentage) dilute hydrochloric acid is slowly added to the reaction solution to acidify the reaction solution to a pH value of 3. NaCl solid is added into the reaction solution to saturate the reaction solution after the temperature of the reaction solution rises to room temperature. The reaction solution is then concentrated three times with ethyl acetate. The organic layers are combined, concentrated, and applied to column chromatography to obtain 3-oxobutanoic acid which is a clear liquid.

In an embodiment of step S3′, 3,4-dibenzyloxyphenylethanol prepared in S1′ and the 3-oxobutanoic acid prepared in S2′ are weighed and placed in a reaction vessel, to which dichloromethane (DCM) is added, followed by ultrasonic dissolving. Then EDCI and DMAP are sequentially added to the mixed solution, which is then reacted at room temperature for 1 hour. Once the completeness of the reaction is confirmed by TLC, the reaction solution is concentrated, and added with saturated sodium chloride water solution, and then extracted with ethyl acetate. The organic layer is concentrated and applied to column chromatography, followed by drying and freezing to obtain 3,4-bis(benzyloxy)phenethyl 3-oxobutanoate.

In an embodiment of step S4′, 3,4-bis(benzyloxy)phenethyl 3-oxobutanoate prepared in S3′ is weighed and placed in a reaction vessel, to which methanol is added, followed by ultrasonic dissolving to form a solution, which is then added with Pd/C under the protection of argon gas, followed by replacing the air in the reaction vessel with hydrogen gas for three times in vacuum. The solution is reacted at room temperature for 2 hours. Once the completeness of the reaction is confirmed by TLC, the reacted solution is filtered with suction to obtain a filtrated liquid, which is concentrated, dried and frozen to obtain 3,4-dihydroxyphenethyl 3-oxobutanoate.

In an embodiment of step S5′, 3,4-dihydroxyphenethyl 3-oxobutanoate prepared in S4′ is weighed and placed in a reaction vessel, to which absolute ethanol is added, followed by ultrasonic dissolving. The reaction vessel is placed in an ice bath at 0° C. After the temperature of the reaction solution is stabilized, NaBH₄ is slowly added portion by portion to react for 15 minutes. Once the completeness of the reaction is confirmed by TLC, anhydrous acetone is added dropwise to the reaction solution to quench the remaining NaBH₄. A solution of saturated hydrochloric acid in ethanol is then added dropwise to the reaction solution, which is then acidified to a pH value of 5, concentrated, and applied to column chromatography to obtain 3,4-dihydroxyphenethyl 3-hydroxybutanoate.

In some embodiments, a ratio of 3,4-dihydroxyphenylethanol, potassium carbonate, anhydrous acetone, and benzyl bromide added in S1′ is 1 mmol to 20 mmol:1 mmol to 100 mmol:1 ml to 200 ml:1 mmol to 80 mmol, and in an embodiment is 6.49 mmol:25.9 mmol:20 ml:13.62 mmol.

In some embodiments, a ratio of ethyl acetoacetate and NaOH water solution in S2′ is 1 ml:3 ml to 10 ml, and in an embodiment is 26 ml:100 ml.

In some embodiments, a ratio of 3,4-dibenzyloxyphenylethanol, 3-oxobutanoic acid, DCM, EDCI, and DMAP in S3′ is 1 mmol to 15 mmol:1 mmol to 60 mmol:1 ml to 200 ml:1 g to 10 g: 1 mg to 1000 mg, and in an embodiment is 5 mmol:10 mmol:30 ml:1.85 g 100 mg.

In some embodiments, a ratio of 3,4-bis(benzyloxy)phenethyl 3-oxobutanoate, methanol, and Pd/C in S4′ is 1 g to 10 g: 1 ml to 300 ml:1 mg to 1000 mg, and in an embodiment is 1.00 g: 25 ml:100 mg, and the pressure of the hydrogen gas is one atmospheric pressure.

In some embodiments, a ratio of 3,4-dihydroxyphenethyl 3-oxobutanoate, absolute ethanol, and NaBH₄ in S5′ is 0.5 g to 5 g: 20 ml to 300 ml:50 mg to 1000 mg, and in an embodiment is 500 mg: 20 ml:95.78 mg.

The present disclosure further provides use of the compound represented by formula I, optically pure isomers of the compound, a mixture of enantiomers of the compound in any ratio, or pharmaceutically acceptable salts of the compound in preparing pharmaceutical compositions such as health foods and drugs for relieving brain fatigue, improving learning or memory ability, or ameliorating mania related to brain fatigue. β-hydroxybutyric acid (and various ester compounds thereof) and hydroxytyrosol (and various derivative ester compounds therefrom) produced by metabolism of the compound work together to significantly relieve brain fatigue.

The present disclosure further provides a pharmaceutical composition, such as a health food or a drug, for relieving brain fatigue, specifically for improving learning and memory abilities, or ameliorating mania related to brain fatigue, including the compound represented by formula I, the optically pure isomer of the compound, the mixture of enantiomers of the compound in any ratio, or the pharmaceutically acceptable salt of the compound.

The present disclosure further provides a method for relieving brain fatigue, the method including administering to a patient in need thereof a therapeutically effective amount of the compound represented by formula I.

In an embodiment, the effective amount for adults is 8.8 mg per kg of body weight per day (the conversion ratio between the dosage of rat and human is 1:0.16).

The applicant utilizes two mitochondrial nutrients, hydroxytyrosol (HT) and β-hydroxybutyric acid (β-HB), to form a novel compound which has a notable effect on amelioration of brain fatigue. The compound can be used in developing new health foods and drugs for relieving brain fatigue. The use of the novel compound for relieving brain fatigue is disclosed for the first time. The amelioration of the brain fatigue is specially embodied in improvement of learning and memory abilities and amelioration of mania.

The effect of the compound on relieving brain fatigue, and specifically on the improvement of learning and memory abilities and amelioration of mania caused by brain fatigue is disclosed for the first time.

The compound has an effect comparable with hydroxytyrosol acetate or ethyl β-hydroxybutyrate taken alone on ameliorating mania, and has an effect better than hydroxytyrosol acetate or ethyl β-hydroxybutyrate taken alone on reducing the decline of learning and memory abilities caused by brain fatigue.

### BRIEF DESCRIPTION OF THE DRAWINGS

FIG. 1A is a graph showing the effects of hydroxytyrosol acetate, ethyl β-hydroxybutyrate, and hydroxytyrosol hydroxybutyrate on the improvement of learning and memory abilities, wherein the abscissa represents the group divided by the ingested drug, and the ordinate represents a swimming time percentage of the rat in the quadrant where a water maze platform is located.

FIG. 1B is a graph showing the effects of hydroxytyrosol acetate, ethyl β-hydroxybutyrate, and hydroxytyrosol hydroxybutyrate on the improvement of learning and memory abilities, wherein the abscissa represents the group divided by the ingested drug, and the ordinate represents a swimming path length percentage of the rat in the quadrant where a water maze test platform is located.

FIG. 2A is a graph showing the effects of hydroxytyrosol acetate, ethyl β-hydroxybutyrate, and hydroxytyrosol hydroxybutyrate on the amelioration of manic mood, wherein the abscissa represents the group divided by the ingested drug, and the ordinate represents the moving speed of the rat.

FIG. 2B is a graph showing the effects of hydroxytyrosol acetate, ethyl β-hydroxybutyrate and hydroxytyrosol hydroxybutyrate on the amelioration of manic mood, wherein the abscissa represents the group divided by the ingested drug, and the ordinate indicates the number of movements of the rat.

FIG. 2C is a graph showing the effects of hydroxytyrosol acetate, ethyl β-hydroxybutyrate, and hydroxytyrosol hydroxybutyrate on the amelioration of manic mood, wherein the abscissa represents the group divided by the ingested drug, and the ordinate indicates the moving path length percentage of the rat in the central area.

FIG. 3 shows ¹H NMR spectrum of β-benzyloxybutyric acid.

FIG. 4 shows ¹H NMR spectrum of 3,4-dibenzyloxyphenylethanol.

FIG. 5 shows ¹H NMR spectrum of 3,4-bis(benzyloxy)phenethyl 3-(benzyloxy)butanoate.

FIG. 6 shows ¹H NMR spectrum of 3,4-bis(benzyloxy)phenethyl 3-oxobutanoate.

FIG. 7 shows ¹H NMR spectrum of 3,4-dihydroxyphenethyl 3-oxobutanoate.

FIG. 8 shows ¹H NMR spectrum of 3,4-dihydroxyphenethyl 3-hydroxybutanoate.

FIG. 9 shows 13C NMR spectrum of 3,4-dihydroxyphenethyl 3-hydroxybutanoate.

FIG. 10 shows HRMS (ESI) spectrum of 3,4-dihydroxyphenethyl 3-hydroxybutanoate.

### DETAILED EMBODIMENTS OF THE DISCLOSURE

The technical solution of the present disclosure will be further described below with reference to specific embodiments and drawings, but it should be understood that the protection scope of the present disclosure is not limited by the specific embodiments.

The present disclosure provides a compound, 3,4-dihydroxyphenethyl 3-hydroxybutanoate (also named hydroxytyrosol hydroxybutyrate), represented by a structural formula I.

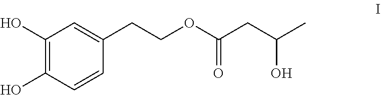 [SMILES: CC(O)CC(=O)OCCc1ccc(O)c(O)c1]

The present disclosure provides an embodiment of a method (method I) for preparing the compound as the following scheme.

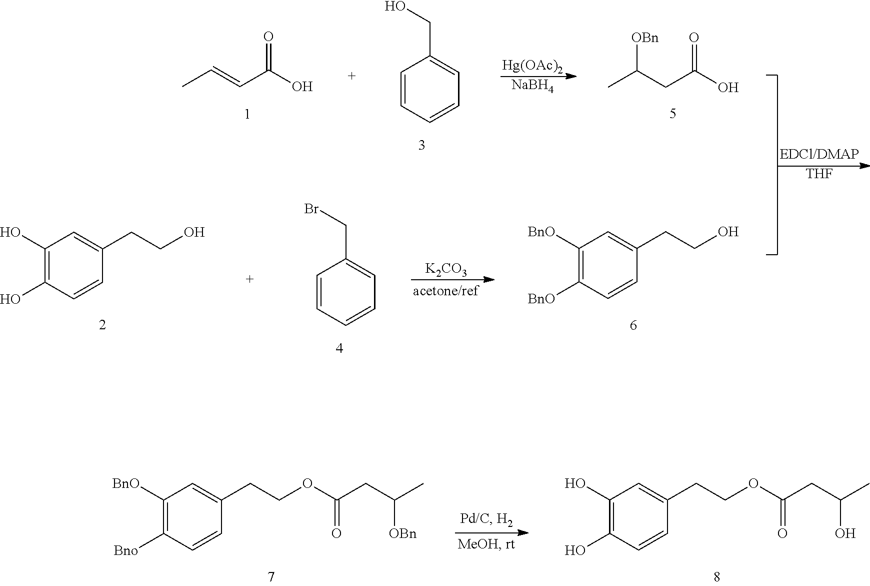

The embodiment of the method I for preparing the compound of formula I includes the following steps.

### S1. Synthesization of β-benzyloxybutyric acid (5)

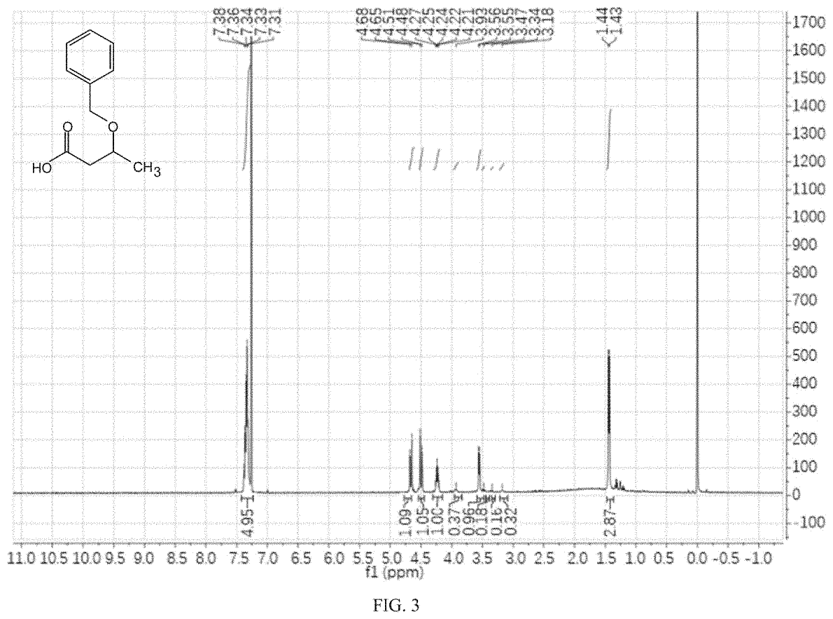

Crotonic acid (1) (2.55 g, 29.7 mmol) was weighed and placed in a 150 ml round-bottom flask, to which benzyl alcohol (3) (30 ml) and mercury acetate (9.63 g, 30 mmol) were sequentially added to form a mixture. The mixture was stirred at room temperature overnight. Then, the flask was placed in a low-temperature condensation tank and cooled to 0° C. 30 ml of sodium hydroxide (3N purity) was added in the flask within 5 to 10 minutes, and then 30 ml of sodium hydroxide (3N purity) water solution containing 0.5 M (0.57 g) of sodium borohydride (NaBH₄) was added in the flask. Then, the mixture was kept at 0° C. for 3 to 10 minutes. After that, the flask was taken out of the tank, and the mixture in the flask was stirred at room temperature for 1 to 2 hours, and filtered to obtain a filtrated liquid, which was extracted with 75 ml ethyl ether 3 to 4 times to remove excess benzyl alcohol (3). The filtrated liquid was then acidified to pH=2 by using 10% hydrochloric acid to precipitate a large amount of white solid, which was filtered out to obtain β-benzyloxybutyric acid (5) as the which solid, 4.32 g, yield 75%. ¹H NMR (400 MHz, CDCl3) δ 7.40-7.28 (m, 5H), 4.58 (dd, J=33.0, 11.6 Hz, 2H), 4.30-4.19 (m, 1H), 3.93-3.18 (m, 1H), 3.55 (d, J=5.5 Hz, 1H), 1.44 (d, J=5.9 Hz, 3H). The ¹H NMR spectrum of β-benzyloxybutyric acid is shown in FIG. 3.

### S2. Synthesization of 3,4-dibenzyloxyphenylethanol (6)

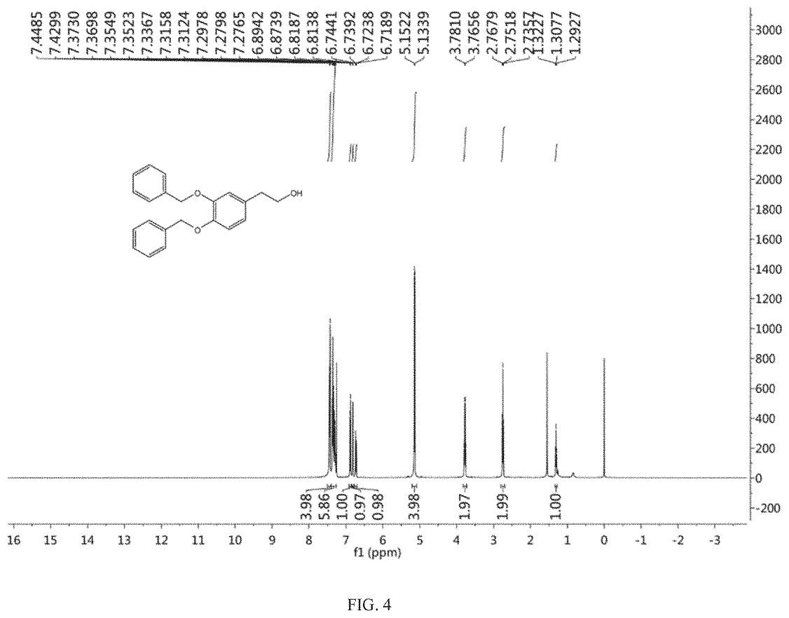

3,4-dihydroxyphenylethanol (2) (1 g, 6.49 mmol) and potassium carbonate (3.59 g, 25.9 mmol) were weighed and mixed in a 50 ml round-bottom flask, to which anhydrous acetone (20 ml) and benzyl bromide (4) (1.62 ml, 13.62 mmol) are sequentially added, followed by stirring at 70° C. under reflux for 4 to 5 hours. Once the completeness of the reaction was confirmed by TLC, the reaction product was filtered to remove the potassium carbonate, and then was concentrated and applied to column chromatography (DCM:EA=20:1) to obtain 3,4-dibenzyloxyphenylethanol (6), 1.86 g, white solid, yield 86%. ¹H NMR (400 MHz, CDCl3) δ 7.45-7.43 (m, 4H), 7.39-7.26 (m, 6H), 6.88 d, J=8.1 Hz, 1H), 6.81 (d, J=2.0 Hz, 1H), 6.73 (dd, J=8.1, 2.0 Hz, 1H), 5.15 (s, 2H), 5.13 (s, 2H), 3.77 (q, J=6.3 Hz, 2H), 2.75 (t, J=6.4 Hz, 2H). The ¹H NMR spectrum of 3,4-dibenzyloxyphenylethanol is shown in FIG. 4.

### S3. Synthesization of 3,4-bis(benzyloxy)phenethyl 3-(benzyloxy)butanoate (7)

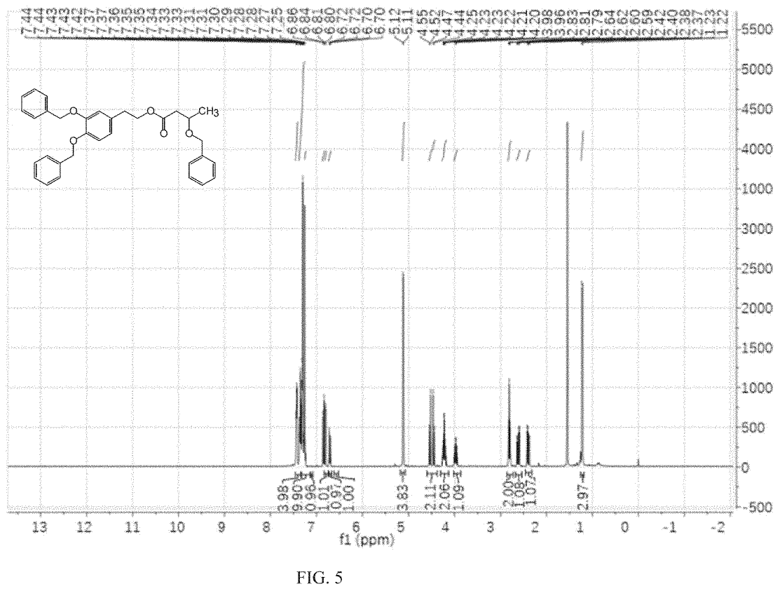

β-benzyloxybutyric acid (5) (1.24 g, 6.4 mmol) was weighed and placed in a 100 ml round-bottom flask, to which THF (45 ml) was added and dissolved. Then, 3,4-dibenzyloxyphenylethanol (6) (1.34 g, 4 mmol) along with EDCI (1.53 g, 8 mmol), DMAP (50 mg) were added in the reaction vessel, and then stirred in an oil bath at 30° C. for 3 to 4 hours to have a reaction. The reaction was terminated once disappearance of 3-benzyloxybutyric acid was confirmed by TLC. The resultant was concentrated, and then dissolved and washed with EA 2 to 3 times to obtain a solution, which was concentrated and applied to column chromatography (PE:DCM=1:1) to obtain 3,4-bis(benzyloxy)phenethyl 3-(benzyloxy)butanoate (7), which is a pink oil, 823 mg, yield 40%. ¹H NMR (400 MHz, CDCl3) δ 7.47-7.39 (m, 4H), 7.38-7.26 (m, 10H), 7.25-7.23 (m, 1H), 6.85 (d, J=8.2 Hz, 1H), 6.80 (d, J=1.9 Hz, 1H), 6.71 (dd, J=8.2, 2.0 Hz, 1H), 5.12 (s, 2H), 5.11 (s, 2H), 4.49 (dd, J=33.0, 11.6 Hz, 2H), 4.28-4.17 (m, 2H), 4.02-3.92 (m, 1H), 2.81 (t, J=7.1 Hz, 2H), 2.61 (dd, J=15.0, 7.3 Hz, 1H), 2.39 (dd, J=15.0, 5.7 Hz, 1H), 1.22 (d, J=6.2 Hz, 3H). The ¹H NMR spectrum of 3,4-bis(benzyloxy)phenethyl 3-(benzyloxy)butanoate is shown in FIG. 5.

### S4. Synthesization of 3,4-dihydroxyphenethyl 3-hydroxybutanoate (8)

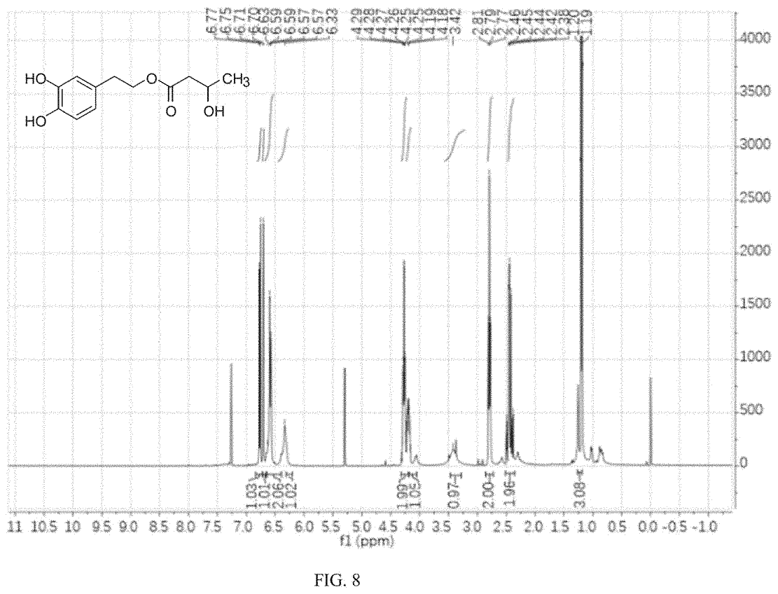

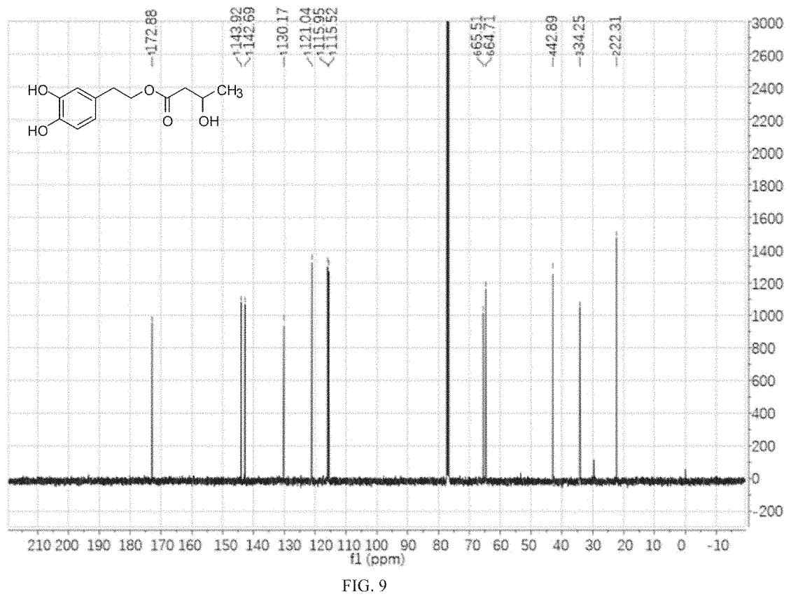

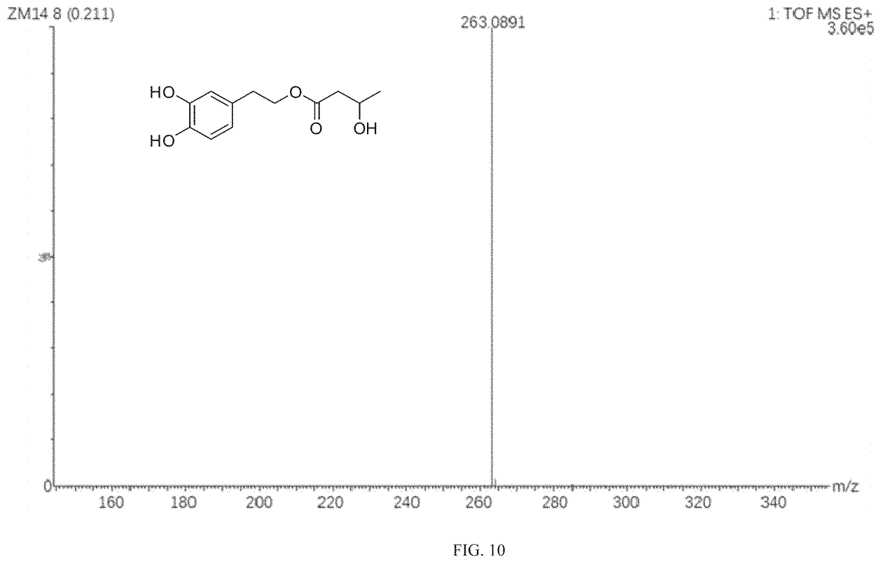

3,4-bis(benzyloxy)phenethyl 3-(benzyloxy)butanoate (7) (2 g, 3.92 mmol) was weighed and placed in a 100 ml round-bottom flask, to which anhydrous methanol (40 ml) was added for dissolving, and then 10% Pd/C (200 mg) was added, followed by stirring at room temperature in hydrogen gas atmosphere (the pressure of the hydrogen gas was one atmospheric pressure) for 16 hours to have a reaction. The reaction product was filtered, concentrated, and applied to column chromatography (DCM:MeOH=80:1) to obtain 3,4-dihydroxyphenethyl 3-hydroxybutanoate (8), which was a colorless or light yellow oil, 762 mg, yield 80%. ¹H NMR (400 MHz, CDCl3) δ 6.76 (d, J=8.0 Hz, 1H), 6.70 (d, J=1.8 Hz, 1H), 6.69-6.51 (m, 2H), 6.33 (s, 1H), 4.32-4.23 (m, 2H), 4.23-4.14 (m, 1H), 3.42 (s, 1H), 2.79 (t, J=6.8 Hz, 2H), 2.48-2.37 (m, 2H), 1.20 (d, J=6.3 Hz, 3H). 13C NMR (101 MHz, CDCl3) δ 172.88, 143.92, 142.69, 130.17, 121.04, 115.95, 115.52, 65.51, 64.71, 42.89, 34.25, 22.31. HRMS(ESI): calculated for C₁₂H₁₆NaO₅⁺[M+Na]⁺, 263.0890; found 263.0891. The ¹H NMR spectrum of 3,4-dihydroxyphenethyl 3-hydroxybutanoate is shown in FIG. 8. The ¹³C NMR spectrum of 3,4-dihydroxyphenethyl 3-hydroxybutanoate is shown in FIG. 9. The HRMS (ESI) spectrum of 3,4-dihydroxyphenethyl 3-hydroxybutanoate is shown as FIG. 10.

The present disclosure further provides another embodiment of the method (method II) for preparing the compound as the following scheme.

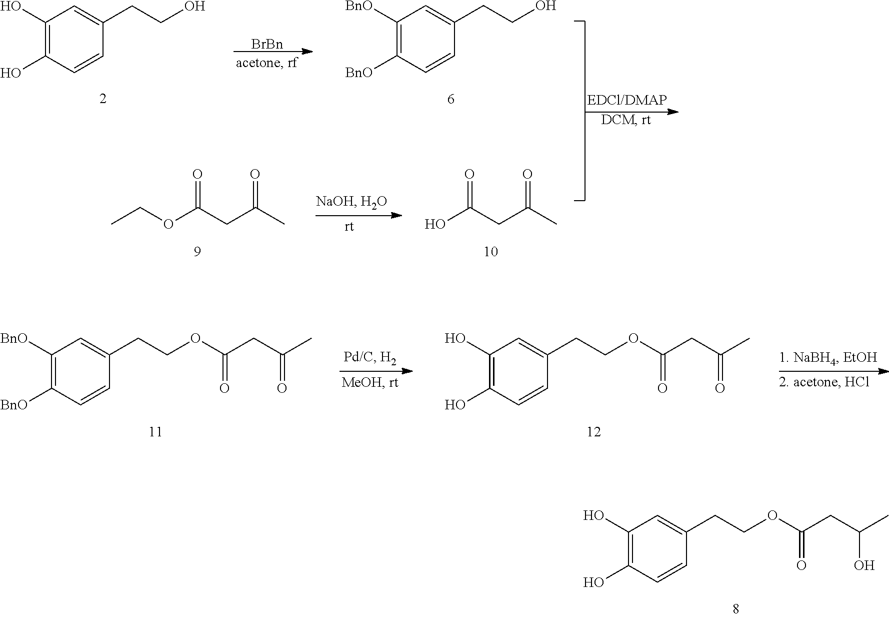

The embodiment of the method II for preparing the compound of formula I includes the following steps.

### S1′. Synthesization of 3,4-dibenzyloxyphenylethanol (6)

3,4-dihydroxyphenylethanol (2) (1 g, 6.49 mmol) and potassium carbonate (3.59 g, 25.9 mmol) were weighed and placed in a 50 ml round-bottom flask, to which anhydrous acetone (20 ml) was added for dissolving, and then benzyl bromide (4) (1.62 ml, 13.62 mmol) was added to form a mixture. The mixture was stirred and reacted at 70° C. under reflux for 4 to 5 hours. Once the completeness of the reaction was confirmed by TLC, potassium carbonate was filtered off, and then the reaction product was concentrated and applied to column chromatography (DCM:EA=20:1) to obtain 3,4-dibenzyloxyphenylethanol (6), 1.86 g, white solid, yield 86%. ¹H NMR (400 MHz, CDCl3) δ 7.45-7.43 (m, 4H), 7.39-7.26 (m, 6H), 6.88 d, J=8.1 Hz, 1H), 6.81 (d, J=2.0 Hz, 1H), 6.73 (dd, J=8.1, 2.0 Hz, 1H), 5.15 (s, 2H), 5.13 (s, 2H), 3.77 (q, J=6.3 Hz, 2H), 2.75 (t, J=6.4 Hz, 2H). The ¹H NMR spectrum of 3,4-dibenzyloxyphenylethanol is shown in FIG. 4.

### S2′. Synthesization of 3-oxobutanoic acid (10)

26 ml of ethyl acetoacetate (6) was weighed and placed in a round-bottom flask, to which 100 ml of freshly prepared NaOH (N purity) water solution was added, followed by reacting in oil bath at 60° C. for 3 hours. Once the completeness of the reaction was confirmed by TLC, the flask was cooled to room temperature and placed in an ice bath environment at 0° C. 10% (mass percentage) of dilute hydrochloric acid was slowly added to the reaction solution to acidify the reaction solution to pH=3, followed by adding NaCl solid to saturate the reaction solution after the temperature of the reaction solution rose to room temperature. The reaction solution was then concentrated three times with ethyl acetate. The organic layers were combined, concentrated, and applied to column chromatography to obtain 3-oxobutanoic acid which was a clear liquid and a waxy solid after freezing, 2.80 g, yield 80%.

### S3′. Synthesization of 3,4-bis(benzyloxy)phenethyl 3-oxobutanoate (11)

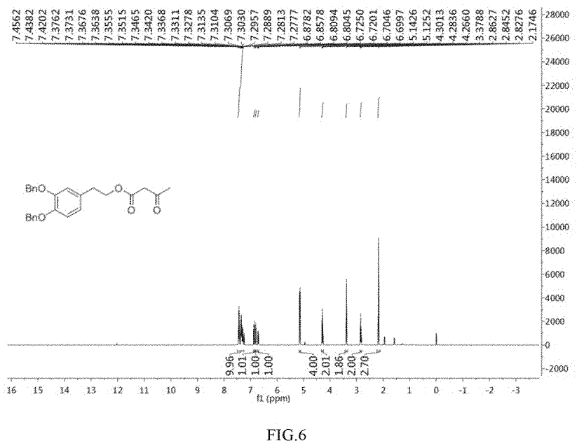

3,4-dibenzyloxyphenylethanol (1.70 g, 5 mmol) and 3-oxobutanoic acid (1.02 g, 10 mmol) were weighed and placed into a round-bottom flask, to which 30 ml of DCM was added, followed by ultrasonic dissolving. Then, 1.85 g of EDCI and 100 mg of DMAP were sequentially added to the mixed solution, which was then reacted at room temperature for 1 hour. Once the completeness of the reaction was confirmed by TLC (developing solvent PE:EA=3:1, Rf=0.7), the reaction solution was concentrated and added with 25 ml of saturated sodium chloride water solution, and then extracted with ethyl acetate. The organic layer was concentrated and applied to column chromatography, followed by drying and freezing to obtain 3,4-bis(benzyloxy)phenethyl 3-oxobutanoate, 1.88 g, waxy off-white solid, yield 88%. ¹H NMR (400 MHz, CDCl3) δ 7.49-7.26 (m, 10H), 6.87 (d, J=8.2 Hz, 1H), 6.81 (d, J=2.0 Hz, 1H), 6.71 (dd, J=8.2, 2.0 Hz, 1H), 5.13 (d, J=7.0 Hz, 1H), 4.28 (t, J=7.0 Hz, 2H), 3.38 (s, 2H), 2.85 (t, J=7.0 Hz, 2H), 2.17 (s, 3H). The ¹H NMR spectrum of 3,4-bis(benzyloxy)phenethyl 3-oxobutanoate is shown in FIG. 6.

### S4′. Synthesization of 3,4-dihydroxyphenethyl 3-oxobutanoate (12)

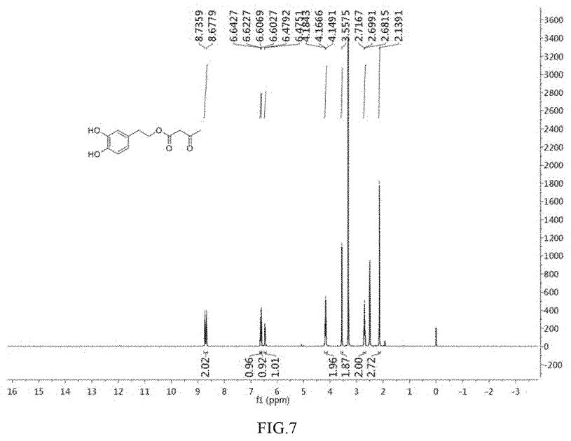

1.00 g of 3,4-bis(benzyloxy)phenethyl 3-oxobutanoate was weighed and placed in a two-neck bottle, to which 25 ml of methanol was added, followed by ultrasonic dissolving to form a solution. 100 mg of Pd/C was added to the solution under the protection of argon gas, followed by replacing the air in the bottle with hydrogen gas for three times in vacuum. The solution was reacted at room temperature for 2 hours. Once the completeness of the reaction was confirmed by TLC, the reacted solution was filtered with suction to obtain a filtrated liquid. The filtrate liquid was concentrated, dried and frozen to obtain 3,4-dihydroxyphenethyl 3-oxobutanoate, 0.54 g, waxy off-white solid, yield 95%. ¹H NMR (400 MHz, DMSO) δ 8.71 (d, J=23.2 Hz, 2H), 6.63 (d, J=8.0 Hz, 1H), 6.60 (d, J=1.7 Hz, 1H), 6.47 (dd, J=7.9, 1.7 Hz, 1H), 4.17 (t, J=7.1 Hz, 2H), 3.56 (s, 2H), 2.70 (t, J=7.0 Hz, 2H), 2.14 (s, 3H). The ¹H NMR spectrum of 3,4-dihydroxyphenethyl 3-oxobutanoate is shown in FIG. 7.

### S5′. Synthesization of 3,4-dihydroxyphenethyl 3-hydroxybutanoate (8)

500 mg of 3,4-dihydroxyphenethyl 3-oxobutanoate was weighed and placed in a 50 ml round-bottom flask, to which absolute ethanol was added, followed by ultrasonic dissolving. The flask was placed in an ice bath at 0° C. After the temperature of the reaction solution was stabilized, 95.78 mg of NaBH₄ was slowly added portion by portion to react for 15 minutes. Once the completeness of the reaction was confirmed by TLC, anhydrous acetone was added dropwise to the reaction solution to quench the remaining NaBH₄. A solution of saturated hydrochloric acid in ethanol was added dropwise to the reaction solution, which was then acidified to pH=5, concentrated, and applied to column chromatography to obtain 3,4-dihydroxyphenethyl 3-hydroxybutanoate, 430 mg, colorless or light yellow oil, yield 85%. ¹H NMR (400 MHz, CDCl3) δ 6.76 (d, J=8.0 Hz, 1H), 6.70 (d, J=1.8 Hz, 1H), 6.69-6.51 (m, 2H), 6.33 (s, 1H), 4.32-4.23 (m, 2H), 4.23-4.14 (m, 1H), 3.42 (s, 1H), 2.79 (t, J=6.8 Hz, 2H), 2.48-2.37 (m, 2H), 1.20 (d, J=6.3 Hz, 3H). 13C NMR (101 MHz, CDCl3) δ 172.88, 143.92, 142.69, 130.17, 121.04, 115.95, 115.52, 65.51, 64.71, 42.89, 34.25, 22.31. HRMS(ESI): calculated for C₁₂H₁₆NaO₅[M+Na]+, 263.0890; found 263.0891. The ¹H NMR spectrum of 3,4-dihydroxyphenethyl 3-hydroxybutanoate is shown as FIG. 8. The ¹³C NMR spectrum of 3,4-dihydroxyphenethyl 3-hydroxybutanoate is shown as FIG. 9. The HRMS (ESI) spectrum of 3,4-dihydroxyphenethyl 3-hydroxybutanoate is shown as FIG. 10.

### Experiments

### 1. Experimental Materials

Hydroxytyrosol acetate (CAS No. 69039-02-7) was purchased from Santa Cruz Biotechnology, Inc.; and ethyl β-hydroxybutyrate (CAS No. 24915-95-5) was purchased from Aladdin Reagent (Shanghai) Co., Ltd.

### 2. Experimental Animal Feeding and Model Establishment

8-week-old adult male SD rats (body weight: 250 g) purchased from Experimental Animal Center of The Second Military Medical University in Shanghai were used in the experiments. The rats were kept in an animal room with controlled temperature (22° C. to 28° C.) and humidity (60%). The light of the room was maintained in a 12 h/12 h day/night light cycle. The rats were able to eat and drink freely during the experiments. A brain fatigue rat model was established by using the sleep deprivation method in the experiments. The experimental rats were divided into five groups, with 10 rats in each group.

The five groups are as follows: (1) a normal feeding group with normal saline intragastrically administered daily (hereinafter referred to as the control group); (2) a brain fatigue model group established by the sleep deprivation method, with normal saline intragastrically administered daily (hereinafter referred to as the brain fatigue group); (3) a brain fatigue model group established by the sleep deprivation method, with 35 mg/kg of hydroxytyrosol acetate intragastrically administered daily (hereinafter referred to as HTac group, as shown in the following table); (4) a brain fatigue model group established by the sleep deprivation method, with 23.6 mg/kg of β-hydroxybutyrate intragastrically administered daily (hereinafter referred to as HBET group, as shown in the following table); (5) a brain fatigue model group established by the sleep deprivation method, with 42.9 mg/kg of hydroxytyrosol hydroxybutyrate intragastrically administered daily (hereinafter referred to as HTHB group, as shown in Table 1).

TABLE 1

| Group | HTac | HBET | HTHB |
|---|---|---|---|
| Compound | hydroxytyrosol | β- | hydroxytyrosol |
| | acetate | hydroxybutyrate | hydroxybutyrate |
| Molecular | 196 | 132 | 240 |
| Weight | | | |
| Dosage for Rat | 35 | 23.6 | 42.9 |
| (mg/Kg/day) | | | |
| Drug | 8.75 mg of the | 5.8 mg of the | 10.725 mg of |
| Preparation¹ | compound | compound | the compound |
| | dissolved | dissolved | dissolved |
| | (or suspended) | (or suspended) | (or suspended) |
| | in 1 ml of water | in 1 ml of water | in 1 ml of water |
| Intragastrical | 1 ml | 1 ml | 1 ml |
| Administration | | | |
| Amount of | | | |
| Drug Per Time | | | |
| (once a day) | | | |

¹The drugs were freshly prepared before use, and the amounts of the compounds were calculated based on 250 g rat body weight.

The rats were preconditioned in the animal room for one week, during which the drugs were intragastrically administered. From the second day of the acquisition training of the water maze experiment, the rats other than the control group were started to be subjected to the small-platform-over-water sleep deprivation to establish the brain fatigue stress model. The sleep deprivation apparatus was mainly composed of two parts: a water tank and a platform disposed in the tank. The tank was filled with water. The size of the platform can only for the hind legs of the rat to stand. The platform was fixed to the bottom of the tank, and the top surface of the platform is above the water surface. The rat was placed on the platform for sleep deprivation. Once the rat fell asleep, its muscle tension would decrease and its center of gravity would move forward, which would cause the rat to fall into the water and suddenly be awakened. In order to avoid this, the rat must keep a slight grip on the edge of the platform, which deprived the sleep of the rat and caused brain fatigue.

### 3. Experimental Methods

1) Water Maze Experiment

In the Morris water maze experiment, a large round black water pool with a diameter of 120 cm and a height of 50 cm was equally divided into four quadrants, I, II, III, and IV on the computer monitor screen. Tap water was injected into the pool to reach a depth of 30 cm. The water pool was placed at a room with a temperature of 26° C.±2° C. and a uniform brightness of 150 l×. Various noticeable visual cues (A4 paper-sized black geometric figures) were placed around the pool. The escape platform with a diameter of 12 cm was placed under the water in the center of the quadrant IV, and the surface of the platform was submerged 2 cm below the water surface. Non-toxic and odorless black dye was added to the water and mixed well with the water to ensure that the swimming rat cannot see the platform. The water in the pool was changed once a day and had the temperature stabilized at 26° C.±2° C.

The water maze experiment includes an acquisition training followed with an exploration test.

The acquisition training of the water maze experiment started from day 1 after the one-week preconditioning of the rats in the animal room. On day 2, the rats were continued to be subjected to the acquisition training and then subjected to the sleep deprivation. On day 3, the sleep-deprived rats were continued to be subjected to the acquisition training and then subjected to the sleep deprivation. On day 4, the sleep-deprived rats were continued to be subjected to the acquisition training and then subjected to the sleep deprivation. On day 5, the sleep-deprived rats were subjected to the exploration test and an open field test, and then subjected to the sleep deprivation. Drug intervention was given continuously to the rats during the experiments.

In the acquisition training, each rat was placed in the quadrants I, II, and III of the water pool every day to subject the training. The order of the quadrants that the rat was placed in each day is shown in Table 2. The rat was placed in the water of one quadrant and faced the pool wall at the beginning of one training test. Once the rat was in the water, it was released and allowed to find the location of the escape platform within 120 seconds. The experimenter would gently guide the rat to the platform if the rat could not find the platform within 120 seconds. Once the rat was on the platform, it was allowed to rest for 10 seconds and to observe the spatial cues around the platform. If the rest time was less than 10 seconds, the rat needed to be guided again. The traveling time and path length that the rat cost for finding the hidden platform was recorded by using a video tracking system. Then, the rat is removed from the water maze as finishing one training test. After the rat finished one training test started from one quadrant, it was placed in and released from another quadrant for another training test. Between two training tests, each rat was allowed to rest for more than five minutes to recover body temperature and physical strength. The order of the starting quadrants in each day was randomly set during the four-day training process and recorded as in Table 2.

TABLE 2
Order of starting quadrants in acquisition training

| Day | First training test | Second training test | Third training test |
|---|---|---|---|
| 1 | Quadrant I | Quadrant II | Quadrant III |
| 2 | Quadrant III | Quadrant I | Quadrant II |
| 3 | Quadrant II | Quadrant III | Quadrant I |
| 4 | Quadrant I | Quadrant III | Quadrant II |

After the three days of the sleep deprivation, the rats were immediately subjected to the exploration test, in which the platform was removed, and the rat was directly placed into the fourth quadrant. The time and path length of the rat swam in the fourth quadrant in 120 seconds and the swimming situation of the rat were recorded. In the exploration test, due to the survival and water avoidance instinct, the rat would try to find the escape platform, which had been placed in the center of the quadrant IV, on the basis of its memory in the acquisition training.

Evaluation indicators: the percentages of the time and path length of the rat swam in the quadrant IV. The higher the percentages, the better the learning and memory abilities of the rat.

2) Open Field Test

The open field test is a classic behavioral test to evaluate emotion of rodents, which is based on the principle that the rodents have instincts to fear an open field and have spontaneous phobotaxis activities. Abnormalities in phobotaxis can indicate emotional abnormalities of rodents. The apparatus of the open field test was mainly composed of a black PVC box and a video tracking system. The box was 80 cm in both length and width and 50 cm in height. A camera of the video tracking system was hung at a height of 1.5 meters from the bottom of the box. The rat can move freely in the open field box, and the camera facing the box recorded the movements of the rat. By using the video tracking system, the interior of the box was divided into 16 squares arranged in 4×4 matrix, in which 12 surrounding squares were defined as a surrounding area, and 4 internal squares were defined as a central area, respectively.

The open field test follows the last day of the water maze experiment. After the water maze experiment, each rat was put back into the cage and rested for 10 minutes. Then in the open field test, the rat was placed at the center of the box bottom in a quiet environment, and was video recorded by the camera for 5 minutes, after which the rat was removed from the box. Before placing another rat, the inner wall and the bottom surface of the box were cleaned to avoid the remaining informational smell and feces of the previous rat from affecting the test results of the next rat. The parameters such as the number of observable movements, moving speed, and the moving path length in each square were recorded and calculated by computer software of the video tracking system.

Evaluation indicators: moving speed, number of movements, and moving path length percentage of the rat in the central area. Manic mood is a manifestation of brain fatigue. Compared with the rats in the control group, experimental rats with brain fatigue would exhibit abnormal excitements, faster movements in the open field, decreased numbers of movements (a tendency to stop moving would decrease), and increased path lengths of movements in the central area of the open field.

3) Statistical Analysis

The experiment results were expressed in form of mean±S.E.M (S.E.M is standard error of mean). The blank group data was statistical results of 15 rats, and each of the other 5 groups of data was statistical results of 10 rats. A few unreasonable data were excluded based on the 99.9% confidence interval. One Way-ANOVA was used for data analysis. The statistical significance p value was represented by *: p<0.05, **: p<0.01, ***: p<0.001, ****: p<0.0001.

### 4. Effect of Hydroxytyrosol Hydroxybutyrate on Improving Learning and Memory in Rats with Brain Fatigue

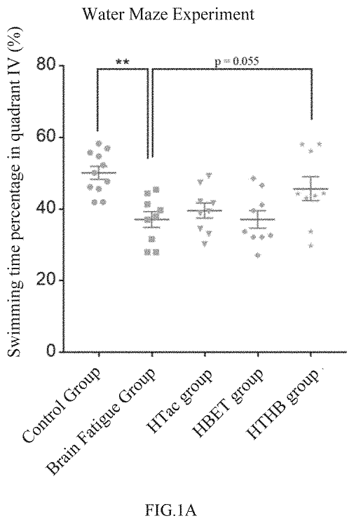

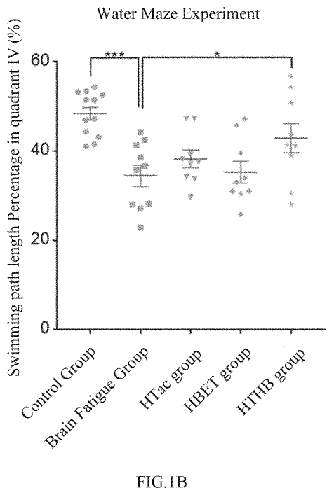

The effect of hydroxytyrosol hydroxybutyrate on the improvement of learning and memory abilities of the rats with brain fatigue was evaluated using the water maze experiment. The effect of brain fatigue caused by sleep deprivation on the learning and memory abilities of the rats and the improvements of learning and memory abilities brought by the compound were revealed by the various indicators of the water maze experiment. The platform was placed under the quadrant IV of the water maze during the acquisition training. After the four-day training, the underwater platform was removed, and the rats were placed in the water pool for a period of time. The percentages of time that the rats spent in the quadrant IV and the percentages of path length that the rats swam in the quadrant IV were recorded. The higher the percentages, the better the learning and memory abilities of the rats. The percentages of the spending time and swimming path length of the rats in the control group, the brain fatigue group, the HTac group, the HBET group, and the HTHB group in the quadrant IV are shown in FIGS. 1A and 1B. The results showed that the swimming time in the quadrant IV of the brain fatigue group was significantly different from that of the control group. Moreover, the swimming time in the quadrant IV of the HTHB group was significantly different from that of the brain fatigue group. The swimming path length in the quadrant IV of the brain fatigue group was significantly different from that of the control group. Moreover, the swimming path length in the quadrant IV of the HTHB group was significantly different from that of the brain fatigue group. Compared with the rats in the control group, the rats in the brain fatigue group had a significant decline in learning and memory abilities. Compared with hydroxytyrosol acetate and ethyl β-hydroxybutyrate, hydroxytyrosol hydroxybutyrate more obviously improved the learning and memory abilities of the rats with brain fatigue.

### 5. Effect of Hydroxytyrosol Hydroxybutyrate on Ameliorating Manic Mood in Rats with Brain Fatigue

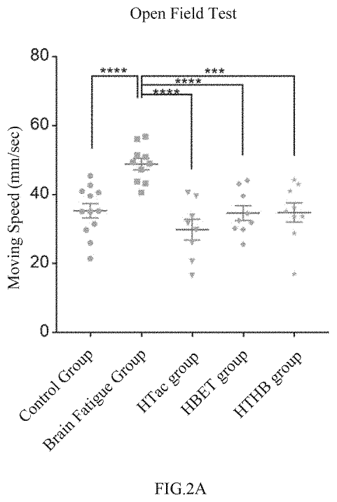

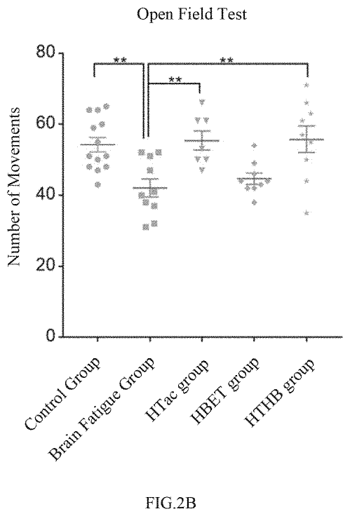

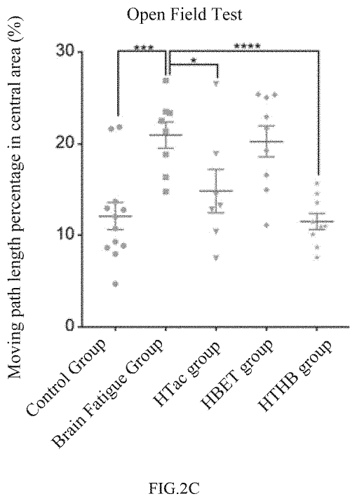

The effect of hydroxytyrosol hydroxybutyrate on ameliorating manic mood of the rats with brain fatigue was evaluated using the open field test. Manic mood was often accompanied with different degrees of depression. The drug ameliorated the manic mood brought by brain fatigue, proving that the compound has the potential to relieve the depression caused by brain fatigue. The effect of brain fatigue on manic mood of the rats and the amelioration brought by the compound was revealed by the various indicators of the open field test. The rats freely moved for a period of time in the square open field. Some of the behavioral indicators in the open field test can intuitively reflect the emotions of the rats. The average moving speeds, the numbers of movements, and the moving path length percentages in the central area in the open field test of the rats in the control group, the brain fatigue group, the HTac group, the HBET group, and the HTHB group were shown in FIGS. 2A, 2B, and 2C, respectively. The rats with manic mood will move faster in the open field, and have lower tendency to stop moving, thus have reduced total numbers of movements, and have higher tendency to move in the central area of the open field rather than in the surrounding area. The results showed that the average moving speed of the brain fatigue group was significantly different from that of the control group. Moreover, the average moving speed of the HTac group, the HBET group, and the HTHB group were significantly different from that of the brain fatigue group. The number of movements of the brain fatigue group was significantly different from that of the control group. Moreover, the number of movements of the HTac group and the number of movements of the HTHB group were significantly different from that of the brain fatigue group. The moving path length percentage in the central area of the brain fatigue group was significantly different from that of the control group. Moreover, the moving path length percentages in the central area of the HTac group and of the HTHB group were significantly different from that of the brain fatigue group. The manic mood of rats with brain fatigue was significantly more intense than that of the control group. However, after administrating hydroxytyrosol acetate, ethyl β-hydroxybutyrate, and hydroxytyrosol hydroxybutyrate respectively, the manic mood of the rats was significantly ameliorated to certain degrees, which showed that hydroxytyrosol hydroxybutyrate inherited the effect of hydroxytyrosol acetate and ethyl β-hydroxybutyrate on the amelioration of manic mood. Moreover, hydroxytyrosol hydroxybutyrate even showed better amelioration of manic mood than hydroxytyrosol acetate and ethyl β-hydroxybutyrate in some indicators.

Finally, it is to be understood that the above-described embodiments are intended to illustrate rather than limit the present disclosure. Variations may be made to the embodiments without departing from the spirit of the present disclosure as claimed. Elements associated with any of the above embodiments are envisioned to be associated with any other embodiments. The above-described embodiments illustrate the scope of the present disclosure but do not restrict the scope of the present disclosure.

1. A compound represented by formula I:

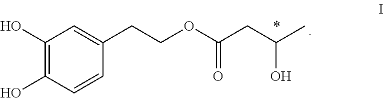 [SMILES: CC(O)CC(=O)OCCc1ccc(O)c(O)c1]

2. A method for preparing the compound of claim 1, the method comprising:
S1, synthesizing β-benzyloxybutyric acid;
S2, synthesizing 3,4-dibenzyloxyphenylethanol;
S3, synthesizing 3,4-bis(benzyloxy)phenethyl 3-(benzyloxy)butanoate; and
S4, synthesizing 3,4-dihydroxyphenethyl 3-hydroxybutanoate.

3. The method of claim 2, wherein the step S1 comprises reacting crotonic acid with benzyl alcohol to synthesize β-benzyloxybutyric acid;
the step S2 comprises reacting 3,4-dihydroxyphenylethanol with benzyl bromide to synthesize 3,4-dibenzyloxyphenylethanol;
the step S3 comprises reacting β-benzyloxybutyric acid synthesized in the step S1 with 3,4-dibenzyloxyphenylethanol synthesized in the step S2 to synthesize 3,4-bis(benzyloxy)phenethyl 3-(benzyloxy)butanoate; and
the step S4 comprises reacting 3,4-bis(benzyloxy)phenethyl 3-(benzyloxy)butanoate synthesized in the step S3 with anhydrous methanol.

4. The method of claim 2, wherein the step S1 comprises:
weighing and placing crotonic acid in a first reaction vessel, to which benzyl alcohol and mercuric acetate are sequentially added to form a first mixture, and stirring the first mixture at room temperature overnight;
cooling the first reaction vessel to about 0° C., adding sodium hydroxide within about 5 minutes to about 10 minutes to the first reaction vessel, then adding a sodium hydroxide water solution containing sodium borohydride to the first reaction vessel, and keeping the first reaction vessel at about 0° C. for about 3 minutes to about 10 minutes;
stirring the first mixture at room temperature for about 1 hour to about 2 hours and then filtering to obtain a filtrated liquid;
extracting the filtrated liquid to remove excess benzyl alcohol; and
acidifying the filtrated liquid to a pH value of about 2 to precipitate β-benzyloxybutyric acid;
the step S2 comprises:
weighing and placing 3,4-dihydroxyphenylethanol and potassium carbonate in a second reaction vessel, to which anhydrous acetone and benzyl bromide are sequentially added to form a second mixture; and
stirring the second mixture at about 70° C. under reflux to react for about 4 hours to about 5 hours;
the step S3 comprises:
weighing and placing β-benzyloxybutyric acid synthesized in the step S1 in a third reaction vessel, to which tetrahydrofuran, 3,4-dibenzyloxyphenylethanol synthesized in the step S2, 1-ethyl-3-(3-dimethylaminopropyl)carbodiimide, and 4-dimethylaminopyridine are added to form a third mixture; and
stirring the third mixture at about 30° C. for about 3 hours to 4 hours;
the step S4 comprises:
weighing and placing 3,4-bis(benzyloxy)phenethyl 3-(benzyloxy)butanoate synthesized in the step S3 in a fourth reaction vessel, to which anhydrous methanol and palladium on carbon catalyst are sequentially added to form a fourth mixture; and
stirring the fourth mixture at room temperature in hydrogen gas atmosphere for about 16 hours.

5. The method of claim 3, wherein a ratio of crotonic acid to benzyl alcohol in the step S1 is 1 mmol to 30 mmol:1 ml to 300 ml;
a ratio of 3,4-dihydroxyphenylethanol to benzyl bromide in the step S2 is 1 mmol to 20 mmol:1 mmol to 80 mmol;
a ratio of β-benzyloxybutyric acid to 3,4-dibenzyloxyphenylethanol in the step S3 is 1 mmol to 15 mmol:1 mmol to 20 mmol; and
a ratio of 3,4-bis(benzyloxy)phenethyl 3-(benzyloxy)butanoate to anhydrous methanol in the step S4 is 1 mmol to 20 mmol:1 ml to 400 ml.

6. The method of claim 3, wherein a ratio of crotonic acid to benzyl alcohol in the step S1 is 29.7 mmol:30 ml;
a ratio of 3,4-dihydroxyphenylethanol to benzyl bromide in the step S2 is 6.49 mmol:13.62 mmol;
a ratio of β-benzyloxybutyric acid to 3,4-dibenzyloxyphenylethanol in the step S3 is 6.4 mmol:4 mmol; and
a ratio of 3,4-bis(benzyloxy)phenethyl 3-(benzyloxy)butanoate to anhydrous methanol in the step S4 is 3.92 mmol:40 ml.

7. A method for preparing the compound of claim 1, the method comprising:
S1′, synthesizing 3,4-dibenzyloxyphenylethanol;
S2′, synthesizing 3-oxobutanoic acid;
S3′, synthesizing 3,4-bis(benzyloxy)phenethyl 3-oxobutanoate;
S4′, synthesizing 3,4-dihydroxyphenethyl 3-oxobutanoate; and
S5′, synthesizing 3,4-dihydroxyphenethyl 3-hydroxybutanoate.

8. The method of claim 7, wherein the step S1′ comprises synthesizing 3,4-dibenzyloxyphenylethanol from 3,4-dihydroxyphenylethanol;
the step S2′ comprises synthesizing 3-oxobutanoic acid from ethyl acetoacetate;
the step S3′ comprises reacting 3,4-dibenzyloxyphenylethanol synthesized in the step S1′ and 3-oxobutanoic acid synthesized in the step S2′ to synthesize 3,4-bis(benzyloxy)phenethyl 3-oxobutanoate;
the step S4′ comprises synthesizing 3,4-dihydroxyphenethyl 3-oxobutanoate from 3,4-bis(benzyloxy)phenethyl 3-oxobutanoate synthesized in the step S3′; and
the step S5′ comprises synthesizing 3,4-dihydroxyphenethyl 3-hydroxybutanoate from 3,4-dihydroxyphenethyl 3-oxobutanoate synthesized in the step in S4′.

9. The method of claim 7, wherein the step S1′ comprises:
weighing and placing 3,4-dihydroxyphenylethanol and potassium carbonate in a first reaction vessel, to which anhydrous acetone and benzyl bromide are sequentially added to form a first mixture; and
stirring the first mixture at about 70° C. under reflux for about 4 hours to about 5 hours;
the step S2′ comprises:
weighing and placing ethyl acetoacetate in a second reaction vessel, to which NaOH water solution is added; and
placing the second reaction vessel in oil bath at about 60° C. for about 3 hours to have a reaction;
the step S3′ comprises:
weighing and placing 3,4-dibenzyloxyphenylethanol synthesized in the step S1′ and the 3-oxobutanoic acid synthesized in the step S2′ in a third reaction vessel, to which dichloromethane, 1-ethyl-3-(3-dimethylaminopropyl)carbodiimide, and 4-dimethylaminopyridine are sequentially added to a mixed solution;
reacting the mixed solution at room temperature for about 1 hour;
the step S4′ comprises:
weighing and placing 3,4-bis(benzyloxy)phenethyl 3-oxobutanoate synthesized in the step S3′ in a fourth reaction vessel, to which methanol is added, followed by ultrasonic dissolving to form a solution;
adding palladium on carbon catalyst to the solution under a protection of argon gas, followed by replacing air in the fourth reaction vessel with hydrogen gas; and
reacting the solution at room temperature for about 2 hours;
the step S5′ comprises:
weighing and placing 3,4-dihydroxyphenethyl 3-oxobutanoate synthesized in the step S4′ in a fourth reaction vessel, to which absolute ethanol is added, followed by ultrasonic dissolving; and
placing the fourth reaction vessel at about 0° C. and adding NaBH₄.

10. The method according to claim 9, wherein a ratio of 3,4-dihydroxyphenylethanol, potassium carbonate, anhydrous acetone, and benzyl bromide in the step S1′ is 1 mmol to 20 mmol:1 mmol to 100 mmol:1 ml to 200 ml:1 mmol to 80 mmol;
a ratio of the ethyl acetoacetate to NaOH water solution in the step S2′ is 1 ml:3 ml to 10 ml;
a ratio of 3,4-dibenzyloxyphenylethanol to 3-oxobutanoic acid in the step S3′ is 1 mmol to 15 mmol:1 mmol to 60 mmol;
a ratio of 3,4-bis(benzyloxy)phenethyl 3-oxobutanoate to methanol in the step S4′ is 1 g to 10 g: 1 ml to 300 ml; and
a ratio of 3,4-dihydroxyphenethyl 3-oxobutanoate, absolute ethanol, and NaBH₄ in the step S5′ is 0.5 g to 5 g: 20 ml to 300 ml:50 mg to 1000 mg.

11. The method according to claim 9, wherein a ratio of 3,4-dihydroxyphenylethanol, potassium carbonate, anhydrous acetone, and benzyl bromide in the step S1′ is 6.49 mmol:25.9 mmol:20 ml:13.62 mmol;
a ratio of the ethyl acetoacetate to NaOH water solution in the step S2′ is 26 ml:100 ml;
a ratio of 3,4-dibenzyloxyphenylethanol to 3-oxobutanoic acid in the step S3′ is 5 mmol:10 mmol;
a ratio of 3,4-bis(benzyloxy)phenethyl 3-oxobutanoate to methanol in the step S4′ is 1.00 g:25 ml; and
a ratio of 3,4-dihydroxyphenethyl 3-oxobutanoate, absolute ethanol, and NaBH₄ in the step S5′ is 500 mg: 20 ml:95.78 mg.

12. Use of the compound of claim 1, an optically pure isomer of the compound, a mixture of enantiomers in any ratio of the compound, a pharmaceutically acceptable salt of the compound, or any combination thereof for preparing a pharmaceutical composition for relieving brain fatigue.

13. A pharmaceutical composition for relieving brain fatigue, comprising the compound of claim 1, an optically pure isomer of the compound, a mixture of enantiomers in any ratio of the compound, a pharmaceutically acceptable salt of the compound, or any combination thereof.

14. The pharmaceutical composition of claim 13 is a health food or a drug.

15. A method for relieving brain fatigue, comprising administering to a patient in need thereof a therapeutically effective amount of the compound of claim 1.

16. The method of claim 15, wherein the effective amount of the compound for an adult is 8.8 mg per kg of body weight per day.

17. The method of claim 15, wherein the relieving brain fatigue comprising improving learning and memory abilities, or ameliorating mania related to brain fatigue.
